(19) United States
(12) Patent Application Publication
(10) Pub. No.: US 2024/0398260 A1
(43) Pub. Date: Dec. 5, 2024

(54) PATIENT PHYSIOLOGICAL MONITOR LOCATION IDENTIFICATION

(71) Applicant: Drägerwerk AG & Co. KGaA, Lübeck (DE)

(72) Inventors: Clifford M. Risher-Kelly, Wells, ME (US); Hans Springensguth, Salem, MA (US); Rajesh S. Rane, Andover, MA (US); Tobias Klotz, Lübeck (DE); Georgios Kokovidis, Waltham, MA (US)

(73) Assignee: Drägerwerk AG & Co. KGaA, Lübeck (DE)

(21) Appl. No.: 18/678,762

(22) Filed: May 30, 2024

Related U.S. Application Data
(60) Provisional application No. 63/470,015, filed on May 31, 2023.

(51) Int. Cl. A61B 5/11; G16H 40/67; H04W 4/029; H04W 4/06

(52) U.S. Cl. CPC A61B 5/1113; G16H 40/67; H04W 4/029; H04W 4/06

(57) ABSTRACT

A mount configured to dock an electronic device, such as a patient monitor, and to broadcast location information thereto. The mount includes a receptacle to removably dock the electronic device. A communication interface only capable of broadcasting signals is utilized to broadcast a signal indicative of a location where the mount is located a limited distance. The mount is not configured for network connectivity and is for use in areas where wired connectivity is not available such as emergency rooms, transport beds, low acuity units and ambulances.

### BACKGROUND

Patient monitors are devices that are configured to receive physiological data associated with a patient from various sensors connected to a patient. A patient monitor may either display a patient's physiological data, monitor a patient's physiological data, or both. A patient monitor may be configured to be worn by a patient, may be a hand-held device, may be docked to or undocked to a mount. Accordingly, a patient monitor may be transportable. A patient monitor may be mounted to a larger unit such as a monitor mount. A monitor mount may be a larger patient monitor or a console that has a docking interface or docking receptacle to which the patient monitor can be removably docked. A monitor mount includes power, network connectivity and/or additional periphery including, for example, displays and ports. A monitor mount has an internet protocol (IP) address assigned thereto and the IP address is associated with a specific location. Alternatively, or in addition, a monitor mount may have a specific location programmed therein. A monitor mount provides an IP address or a programmed location to a patient monitor docked thereto so the patient monitor is associated with a specific location. The specific location for the patient monitor can be used by the patient monitor to couple to other devices located at the same location or to adjust different parameters such as data collected, data transmitted, alarm status, and mode of operation.

A monitor mount requires power and network connectivity and may provide the same to a patient monitor. As some locations, such as emergency rooms, transport beds, low acuity units and ambulances, may not have power and/or network connectivity monitor mounts are not available in these locations. These locations may include a simple mount that secures a patient monitor to a device such as a bed rail, IV pole, or a display (e.g., VESA) mount located therein to secure the patient monitor. A simple mount does not include a network connection and therefore does not have an IP address or an ability to provide a location to a patient monitor. Accordingly, in these locations a patient monitor does not know its location and cannot be coupled to other devices at the location or automatically adjust parameters based on the location.

Thus, a system that enables location information to be provided to a patient monitor at locations where network connections and possibly power are not available may be desirable.

### SUMMARY

One or more embodiments provide a mount configured to dock an electronic device and provide location information thereto. The mount includes a receptacle, a communication interface and a power source. The receptacle is to removably dock the electronic device. The communication interface is only capable of broadcasting, and broadcasts a signal indicative of a location where the mount is located. The signal is only broadcast a limited distance. The mount is not configured for network connectivity.

One or more embodiments provide a method for providing a patient monitor with location information in a location where wired network connectivity is not available. The method includes obtaining a patient monitor mount having a communication interface only capable of broadcasting signals and securing the mount to a physical device in the location. A signal indicative of the location is programmed in the communication interface. The signal indicative of the location is broadcast a limited distance. The patient monitor is docked to the mount and receives the signal broadcast from the mount. The patient monitor assigns a location identification thereto based on the signal.

One or more embodiments provide a patient monitoring system. The system includes a mount and a patient monitor. The mount is not configured for network connectivity and includes a receptacle for removable docking, a Bluetooth module only capable of broadcasting signals, wherein the communication interface broadcasts a signal indicative of a location where the mount is located a limited distance; and a power source. The patient monitor is capable of being docked in the mount and includes a processor, a communication interface to provide communications with a network, wherein the communication interface includes a Bluetooth module configured to receive the broadcast signals; a plurality of sensors to monitor various physiological parameters of the patient; and a display to display at least a subset of the physiological parameters, wherein the patient monitor associates itself with a location based on the signal indicative of a location received and provides the location to a server for further processing.’

The following are additional aspects of the invention.

Aspect 1: A method of associating a location with a patient monitor having a monitor housing, a graphical user interface, a power source, a communication interface capable of communicating using a wireless communication protocol, and a physiological sensor interface, the method comprising:
(a) providing a first mount having a first mount housing, first cradle, a first mount power source, and a first wireless communication interface, the first cradle being adapted to removably secure the patient monitor;
(b) broadcasting a first location signal using the first wireless communication interface, the first location signal having a signal strength of no more than −98 dBm at a distance of 10 cm from the first mount housing, the first location signal including location data that is associated with a first location and the first mount by a hospital network; and
(c) associating the patient monitor with the first location when the patient monitor receives the first location signal.

Aspect 2: The method of aspect 1, further comprising:
(d) pairing the first mount with the patient monitor using a second wireless communication interface.

Aspect 3: The method of aspect 2, wherein the second communication interface uses an electromagnetic induction protocol.

Aspect 4: The method of aspect 3, wherein the electromagnetic induction protocol is selected from the group of RFID and NFC.

Aspect 5: The method of aspect 1, wherein step (c) further comprises communicating the received first location signal to the hospital network, wherein the patient monitor is associated with the first location by the hospital network.

Aspect 6: The method of aspect 1, further comprising:
(e) after performing step (c), initiating at least one location-based action based on the first location.

Aspect 7: The method of aspect 6, wherein the at least one at least one location-based action is selected from the group of: adjusting an operating parameter of the patient monitor, initiating a clinical action for a patient associated with the patient monitor, turning off an alarm, and setting a patient category.

Aspect 8: The method of aspect 1, further comprising:
(f) after performing step (c), disassociating the patient monitor with the first location when the first location signal is not received by the patient monitor.

Aspect 9: The method of aspect 1, further comprising:
(g) after performing step (c), disassociating the patient monitor with the first location when the first location signal is not received by the patient monitor for a first predetermined period of time.

Aspect 10: The method of aspect 1, further comprising, performing step (b) at a predetermined time interval.

Aspect 11: The method of aspect 1, wherein the first location is selected from the group of: a room number, a department of a health care facility, an ambulance number, or a predefined patient movement status.

Aspect 12: The method of aspect 1, wherein step (c) comprises associating the patient monitor with the first location when the patient monitor receives the first location signal a predetermined number of times, the predetermined number of times being greater than one.

Aspect 13: The method of aspect 1, further comprising:
(h) providing electrical power from the first mount to the patient monitor through a plurality of first mount power contacts located within the first cradle that are adapted to engage monitor power contacts located on the monitor housing when the patient monitor is mounted in the first cradle.

Aspect 14: The method of aspect 1, wherein the first location signal has a signal strength of no more than −98 dBm at a distance of 5 cm from the first mount housing.

Aspect 15: The method of aspect 1, further comprising:
(i) providing a second mount having a second mount housing, second cradle, a second power source, and a second wireless communication interface, the second cradle being adapted to removably secure the patient monitor;
(j) broadcasting a second location signal using the second wireless communication interface, the second location signal having a signal strength of no more than −98 dBm at a distance of 10 cm from the second mount housing, the second location signal including location data that is associated with a second location by the hospital network; and
(k) associating the patient monitor with the second location when the patient monitor receives the second location signal.

Aspect 16: A mount for a patient monitor having a monitor housing, a graphical user interface, a power source, at least one communication interface capable of communicating using a wireless communication protocol, and a physiological sensor interface, the mount comprising:
a mount housing having a cradle formed therein adapted to removably receive the patient monitor;
a power connector adapted to supply power to a plurality of power contacts located in the cradle; and
a first wireless communication interface adapted to transmit a location signal having a signal strength of no more than −98 dBm at a distance of 10 cm from the mount housing, the location signal comprising location data indicative of a location of the mount.

Aspect 17: The mount of aspect 16, wherein the location signal has a signal strength of no more than −98 dBm at a distance of 5 cm from the mount housing.

Aspect 18: The mount of aspect 16, wherein the wireless communication interface is adapted to transmit the location signal at a predetermined time interval.

Aspect 19: The mount of aspect 18, wherein the predetermined time interval is no more than 30 seconds.

Aspect 20: The mount of aspect 18, wherein the predetermined time interval is no more than 15 seconds.

Aspect 21: The mount of aspect 18, wherein the predetermined time interval is no less than 1 second.

Aspect 22: The mount of aspect 16, wherein the first wireless communication interface is adapted to transmit a signal using a Bluetooth wireless communication protocol.

Aspect 23: The mount of aspect 16, further comprising a second wireless communication interface using an electromagnetic induction protocol.

Aspect 24: The mount of claim 23, wherein the electromagnetic induction protocol is selected from the group of RFID and NFC.

Aspect 25: The mount of aspect 23, wherein the second wireless interface is adapted to pair with the at least one communication interface of the patient monitor, thereby enabling communication between the patient monitor and the first wireless interface.

Aspect 26: The mount of aspect 16, further comprising a support fastener adapted to removably secure the mount to a bed rail or IV pole.

Aspect 27: A method of associating a location with a patient monitor having a monitor housing, a graphical user interface, a power source, a first monitor communication interface, a second monitor communication interface, and a physiological sensor interface, the method comprising:
(a) providing a first mount having a first mount housing, first cradle, a first mount power source, a first mount wireless communication interface, and a second mount wireless communication interface, the first cradle being adapted to removably secure the patient monitor, the first mount wireless communication interface and the first monitor communication interface using a first wireless communication protocol, the second mount wireless communication interface and the second monitor communication interface using a second wireless communication protocol, the second wireless communication protocol being an electromagnetic induction communication protocol;
(b) broadcasting a first location signal at a first predetermined interval using the first wireless communication interface, the first location signal including location data that is associated with a first location and the first mount by a hospital network;
(c) detecting that the patient monitor is docked to the first cradle;
(d) when the patient monitor is detected as being docked in the first cradle, pairing the first mount communication interface with the first monitor communication interface using the second wireless communication protocol; and
(e) associating the patient monitor with the first location if the location data is received by the first monitor communication interface from the first mount communication interface a first predetermined number of times.

Aspect 28: The method of aspect 27, further comprising:
(f) disassociating the patient monitor from the first location if, after performing step (e), the first location signal is not detected by the first monitor communication interface a second predetermined number of times.

Aspect 29: The method of aspect 27, further comprising:
(g) disassociating the patient monitor from the first location if, after performing step (e), the patient monitor is detected as being undocked from the first cradle.

Aspect 30: The method of aspect 27, wherein the first communication protocol is a Bluetooth communication protocol.

Aspect 31: The method of aspect 27, wherein the second communication protocol is selected from the group of RFID and NFC.

Aspect 32: The method of aspect 27, wherein the patient monitor is detected as being docked to the first cradle when an output voltage of a first hall effect sensor reaches a first predetermined docking voltage.

Aspect 33: The method of aspect 32, wherein the patient monitor is detected as being undocked from the first cradle when the output voltage of the first hall effect sensor drops below a second predetermined docking voltage.

Aspect 34: The method of aspect 27, wherein the first location signal has a signal strength of no more than −98 dBm at a distance of 10 cm from the first mount housing.

### BRIEF DESCRIPTION OF THE DRAWINGS

In the drawings, like reference numbers generally indicate identical, functionally similar, and/or structurally similar elements.

FIG. 1 shows a physiological monitoring device (i.e., patient monitor) according to one or more embodiments;

FIG. 2 illustrates a simple block diagram of components utilized to secure a patent monitor in close proximity to a patient so the patient monitor is not damaged (i.e., does not fall) according to at least one embodiment;

FIG. 3 illustrates a mount for use in locations where wired network connectivity and possibly power are not available according to at least one embodiment;

FIG. 4 illustrates a room in a health care environment having a plurality of beds according to at least one embodiment.

FIG. 5 is a flow diagram showing exemplary steps for programming and use of the simple mount.

FIG. 6 is a flow diagram showing exemplary steps for detecting and using location information by a patient monitor and the hospital network.

FIG. 7 is a perspective view of an exemplary simple mount and patient monitor.

### DETAILED DESCRIPTION

In the following, details are set forth to provide a more thorough explanation of the embodiments. However, it will be apparent to those skilled in the art that embodiments may be practiced without these specific details. In other instances, well-known structures and devices are shown in block diagram form or in a schematic view rather than in detail in order to avoid obscuring the embodiments. In addition, features of the different embodiments described hereinafter may be combined with each other, unless specifically noted otherwise. For example, variations or modifications described with respect to one of the embodiments may also be applicable to other embodiments unless noted to the contrary.

Further, equivalent or like elements or elements with equivalent or like functionality are denoted in the following description with equivalent or like reference numerals. As the same or functionally equivalent elements are given the same reference numbers in the figures, a repeated description for elements provided with the same reference numbers may be omitted. Hence, descriptions provided for elements having the same or like reference numbers are mutually exchangeable.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

In the present disclosure, expressions including ordinal numbers, such as “first”, “second”, and/or the like, may modify various elements. However, such elements are not limited by the above expressions. For example, the above expressions do not limit the sequence and/or importance of the elements. The above expressions are used merely for the purpose of distinguishing an element from the other elements. For example, a first box and a second box indicate different boxes, although both are boxes. For further example, a first element could be termed a second element, and similarly, a second element could also be termed a first element without departing from the scope of the present disclosure.

Directional terminology, such as “top”, “bottom”, “below”, “above”, “front”, “behind”, “back”, “leading”, “trailing”, etc., may be used with reference to the orientation of the figures being described. Because parts of embodiments can be positioned in a number of different orientations, the directional terminology is used for purposes of illustration. It is to be understood that other embodiments may be utilized, and structural or logical changes may be made without departing from the scope defined by the claims. The following detailed description, therefore, is not to be taken in a limiting sense. Directional terminology used in the claims may aid in defining one element's spatial or positional relation to another element or feature, without being limited to a specific orientation.

Instructions may be executed by one or more processors, such as one or more central processing units (CPU), digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein refers to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements. A “controller,” including one or more processors, may use electrical signals and digital algorithms to perform its receptive, analytic, and control functions, which may further include corrective functions. Thus, a controller is a specific type of processing circuitry, comprising one or more processors and memory, that implements control functions by way of generating control signals.

A sensor refers to a component which converts a physical quantity to be measured to an electric signal, for example, a current signal or a voltage signal. The physical quantity may for example comprise electromagnetic radiation (e.g., photons of infrared or visible light), a magnetic field, an electric field, a pressure, a force, a temperature, a current, or a voltage, but is not limited thereto. A magnetic field sensor, for example, includes one or more magnetic field sensor elements that measure one or more characteristics of a magnetic field (e.g., an amount of magnetic field flux density, a field strength, a field angle, a field direction, a field orientation, etc.). In the embodiments that follow, the magnetic field is produced by one or more magnets. However, a current-carrying conductor (e.g., a wire) also generates a magnetic field and can also be a magnetic field source. Each magnetic field sensor element is configured to generate a sensor signal (e.g., a voltage signal) in response to one or more magnetic fields impinging on the sensor element. Thus, a sensor signal is indicative of the magnitude and/or the orientation of the magnetic field impinging on the sensor element.

Magnetic field sensor elements include magneto-resistive sensor elements, inductive sensor elements, and Hall-effect sensor elements (Hall sensor elements), for example, and are mutually exchangeable in the embodiments provided herein. According to one or more embodiments, a plurality of magnetic field sensor elements and a sensor circuitry may be both accommodated (i.e., integrated) in the same chip. The sensor circuit may be referred to as a signal processing circuit and/or a signal conditioning circuit that receives one or more signals (i.e., sensor signals) from one or more magnetic field sensor elements in the form of raw measurement data and derives, from the sensor signal, a measurement signal or sensor data that represents the magnetic field and/or the detection thereof.

Signal conditioning, as used herein, refers to manipulating an analog signal in such a way that the signal meets the requirements of a next stage for further processing. Signal conditioning may include converting from analog to digital (e.g., via an analog-to-digital converter), amplification, filtering, converting, biasing, range matching, isolation and any other processes required to make a sensor output suitable for processing after conditioning.

Thus, the sensor circuit may include an analog-to-digital converter (ADC) that converts the analog signal from the one or more sensor elements to a digital signal. The sensor circuit may also include a DSP that performs some processing on the digital signal, to be discussed below. Therefore, a chip, which may also be referred to as an integrated circuit (IC), may include a circuit that conditions and amplifies the small signal of one or more magnetic field sensor elements via signal processing and/or conditioning.

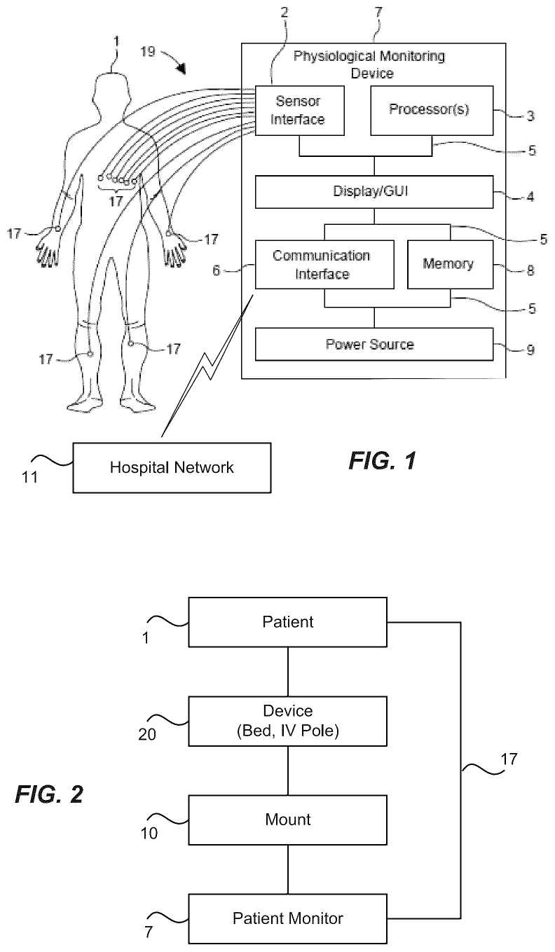

FIG. 1 shows a physiological monitoring device (i.e., patient monitor) according to one or more embodiments. As shown in FIG. 1, the patient monitor 7 is capable of receiving physiological data from various sensors 17 connected to a patient 1. In general, it is contemplated by the present disclosure that the patient monitor 7 includes electronic components and/or electronic computing devices operable to receive, transmit, process, store, and/or manage patient data and information associated performing the functions of the system, which encompasses any suitable processing device adapted to perform computing tasks consistent with the execution of computer-readable instructions stored in a memory or a computer-readable recording medium.

Further, any, all, or some of the computing devices in the patient monitor 7 may be adapted to execute any operating system, including Linux, UNIX, Windows Server, etc., as well as virtual machines adapted to virtualize execution of a particular operating system, including customized and proprietary operating systems. The patient monitor 7 is further equipped with components to facilitate communication with other computing devices over one or more network connections, which may include connections to local and wide area networks, wireless and wired networks, public and private networks, and any other communication network enabling communication in the system.

As shown in FIG. 1, the patient monitor 7 is implemented to monitor various physiological parameters of the patient 1 via the sensors 17. The patient monitor 7 includes a sensor interface 2, one or more processors 3, a display/GUI 4, a communication interface 6, a memory 8, and a power source 9. The sensor interface 2 can be implemented in hardware or combination of hardware and software and is used to connect via wired and/or wireless connections 19 to one or more sensors 17 for gathering physiological data from the patient 1. The sensors 17 may be physiological sensors and/or medical devices configured to measure one or more of the physiological parameters and output the measurements via a corresponding one or more connections 19 to the sensor interface 2. Thus, the connections 19 represent one or more wired or wireless communication channels configured to at least transmit sensor data from a corresponding sensor 17 to the sensor interface 2.

By way of example, sensors 17 may include electrodes that attach to the patient 1 for reading electrical signals generated by or passed through the patient 1. Sensors 17 may be configured to measure vital signs, measure electrical stimulation, measure brain electrical activity such as in the case of a electroencephalogram (EEG), measure blood oxygen saturation fraction from absorption of light at different wavelengths as it passes through a finger, measure a CO2 level and/or other gas levels in an exhalation stream using infrared spectroscopy, measure oxygen saturation on the surface of the brain or other regions, measure cardiac output from invasive blood pressure and temperature measurements, measure induced electrical potentials over the cortex of the brain, measure blood oxygen saturation from an optical sensor coupled by fiber to the tip of a catheter, and/or measure blood characteristics using absorption of light.

The data signals from the sensors 17 include, for example, sensor data related to an electrocardiogram (ECG), non-invasive peripheral oxygen saturation (SpO2), non-invasive blood pressure (NIBP), body temperature, tidal carbon dioxide (etCO2), apnea detection, and/or other physiological data, including those described herein. The one or more processors 3 are used for controlling the general operations of the patient monitor 7, as well as processing sensor data received by the sensor interface 2. Each one of the one or more processors 3 can be, but are not limited to, a central processing unit (CPU), a hardware microprocessor, a multi-core processor, a single core processor, a field programmable gate array (FPGA), a microcontroller, an application specific integrated circuit (ASIC), a digital signal processor (DSP), or other similar processing device capable of executing any type of instructions, algorithms, or software for controlling the operation and performing the functions of the patient monitor 7.

The display/GUI 4 is configured to display various patient data, sensor data, and hospital or patient care information, and includes a user interface implemented for allowing interaction and communication between a user and the patient monitor 7. The display/GUI 4 includes, but is not limited to, a keyboard, a liquid crystal display (LCD), cathode ray tube (CRT) display, thin film transistor (TFT) display, light-emitting diode (LED) display, high definition (HD) display, or other similar display device that may include touch screen capabilities. The display/GUI 4 also provides a means for inputting instructions or information directly to the patient monitor 7. The patient information displayed can, for example, relate to the measured physiological parameters of the patient 1 (e.g., blood pressure, heart related information, pulse oximetry, respiration information, etc.) as well as information related to the transporting of the patient 1 (e.g., transport indicators).

The communication interface 6 enables the patient monitor 7 to directly or indirectly communicate with one or more computing networks and devices, including one or more sensors 17, workstations, consoles, computers, monitoring equipment, alert systems, and/or mobile devices (e.g., a mobile phone, tablet, or other hand-held display device). In a hospital setting, the computing network could be a hospital network 11 that is used to gather and manage patient information, as well as to control devices (such as the patient monitor) use in patient case. The communication interface 6 can include various network cards, interfaces, communication channels, cloud, antennas, and/or circuitry to enable wired and wireless communications with such computing networks and devices. The communication interface 6 can be used to implement, for example, a Bluetooth connection, a cellular network connection, and/or a Wi-Fi connection with such computing networks and devices. Example wireless communication connections implemented using the communication interface 6 include wireless connections that operate in accordance with, but are not limited to, IEEE802.11 protocol, a Radio Frequency For Consumer Electronics (RF4CE) protocol, and/or IEEE802.15.4 protocol (e.g., ZigBee protocol). In essence, any wireless communication protocol may be used.

Additionally, the communication interface 6 can enable direct (i.e., device-to-device) communications (e.g., messaging, signal exchange, etc.) using, for example, a universal serial bus (USB) connection or other communication protocol interface. The communication interface 6 can also enable direct device-to-device connection to other device such as to a tablet, computer, or similar electronic device; or to an external storage device or memory.

The memory 8 can be a single memory device or one or more memory devices at one or more memory locations that include, but is not limited to, a random access memory (RAM), a memory buffer, a hard drive, a database, an erasable programmable read only memory (EPROM), an electrically erasable programmable read only memory (EEPROM), a read only memory (ROM), a flash memory, hard disk, various layers of memory hierarchy, or any other non-transitory computer readable medium. The memory 8 can be used to store any type of instructions and patient data associated with algorithms, processes, or operations for controlling the general functions and operations of the patient monitor 7.

The power source 9 may include a self-contained power source such as a battery pack and/or include an interface to be powered through an electrical outlet (either directly or by way of a mount). The power source 9 can also be a rechargeable battery that can be detached allowing for replacement. In the case of a rechargeable battery, a small built-in back-up battery (or super capacitor) can be provided for continuous power to be provided to the patient monitor 7 during battery replacement. Communication between the components of the patient monitor 7 (e.g., components 2, 3, 4, 6, 8, and 9) are established using an internal bus 5.

Accordingly, the patient monitor 7 is attached to one or more of several different types of sensors 17 configured to measure and readout physiological data related to the patient 1 (e.g., as shown on the left side of FIG. 1). One or more sensors 17 may be attached to patient monitor 7 by, for example, a wired connection coupled to the sensor interface 2. Additionally, or alternatively, one or more sensors 17 may be a wireless sensor that is communicatively coupled to the patient monitor 7 via the communication interface 6, which includes circuitry for receiving data from and sending data to one or more devices using, for example, a Wi-Fi connection, a cellular network connection, and/or a Bluetooth connection. The data signals from the sensors 17 received by the patient monitor 7 may include sensor data related to, for example, body temperature (BT), pulse (heart rate (HR)), and breathing rate (respiratory rate) (RR), an ECG, SpO2, NIBP, and/or etCO2.

The data signals received from the sensors, including an ECG sensor and an SpO2 sensor, can be analog signals. For example, the data signals for the ECG and the SpO2 are input to the sensor interface 2, which can include an ECG data acquisition circuit and an SpO2 data acquisition circuit. Both the ECG data acquisition circuit and the SpO2 data acquisition circuit may include amplifying and filtering circuitry as well as analog-to-digital (A/D) circuitry that converts the analog signal to a digital signal using amplification, filtering, and A/D conversion methods. In the event that the ECG sensor and the SpO2 sensor are wireless sensors, the sensor interface 2 may receive the data signals from a wireless commination module. Thus, a sensor interface is a component configured to interface with one or more sensors 17 and receive sensor data therefrom.

As another example, the data signals related to NIBP, body temperature, and etCO2 can be received from sensors 17 to the sensor interface 2, which can include a physiological parameter interface such as serial interface circuitry for receiving and processing the data signals related to NIBP, temperature, and etCO2. In FIG. 1, the ECG data acquisition circuit, an SpO2 data acquisition circuit, and physiological parameter interface are described as part of the sensor interface 2. However, it is contemplated by the present disclosure that the ECG data acquisition circuit, the SpO2 data acquisition circuit, and physiological parameter interface can be implemented as circuits separate from the sensor interface 2. In the event that the NIBP sensor, the temperature sensor, and the etCO2 sensor are wireless sensors, the sensor interface 2 may receive the data signals from a wireless communication module.

The processing performed by the ECG data acquisition circuit, the SpO2 data acquisition circuit, and external physiological parameter interface may generate analog data waveforms or digital data waveforms that are analyzed by a microcontroller. The microcontroller may be one of the processors 3. The microcontroller, for example, analyzes the digital waveforms to identify certain digital waveform characteristics and threshold levels indicative of conditions (abnormal and normal) of the patient 1 using one or more monitoring methods. A monitoring method may include comparing an analog or a digital waveform characteristic or an analog or digital value to one or more threshold values and generating a comparison result based thereon. The microcontroller is, for example, a processor, an FPGA, an ASIC, a DSP, a microcontroller, or similar processing device. The microcontroller includes a memory or uses a separate memory 8. The memory is, for example, a RAM, a memory buffer, a hard drive, a database, an EPROM, an EEPROM, a ROM, a flash memory, a hard disk, or any other non-transitory computer readable medium.

The memory stores software or algorithms with executable instructions and the microcontroller can execute a set of instructions of the software or algorithms in association with executing different operations and functions of the patient monitor 7 such as analyzing the digital data waveforms related to the data signals from the sensors 17.

FIG. 2 illustrates a block diagram of components utilized to secure the patient monitor 7 in close proximity to the patient 1. The patient 1 is at a location where some type of service (i.e., diagnostics, treatment, procedure) is being performed. A support 20 may be located in close proximity to the patient that can be utilized to secure a mount 10 thereto. The support 20 may be, for example, a rail of the bed that the patient 1 is lying on, an IV pole next to a bed, a chair the patient 1 is on to provide IV fluid to the patient 1, or a display (i.e., VESA) mount located on a pole or a wall in close proximity to the patient 1.

The mount 10 includes a mounting or docking interface that enables the patient monitor 7 to be detachably secured thereby. In this regard, “detachably secure” means that the mount 10 can receive and secure the patient monitor 7, but the patient monitor 7 can also be removed or undocked from the mount 10 by a user when desired. In other words, the patient monitor 7 can be removably docked or removably mounted to the mount 10. In this way, the mount 10 may include a mounting or docking receptacle for receiving the patient monitor 7 therein as part of its mounting or docking interface. Various sensors 17 are utilized by the patient monitor 7 to capture information about the patient 1.

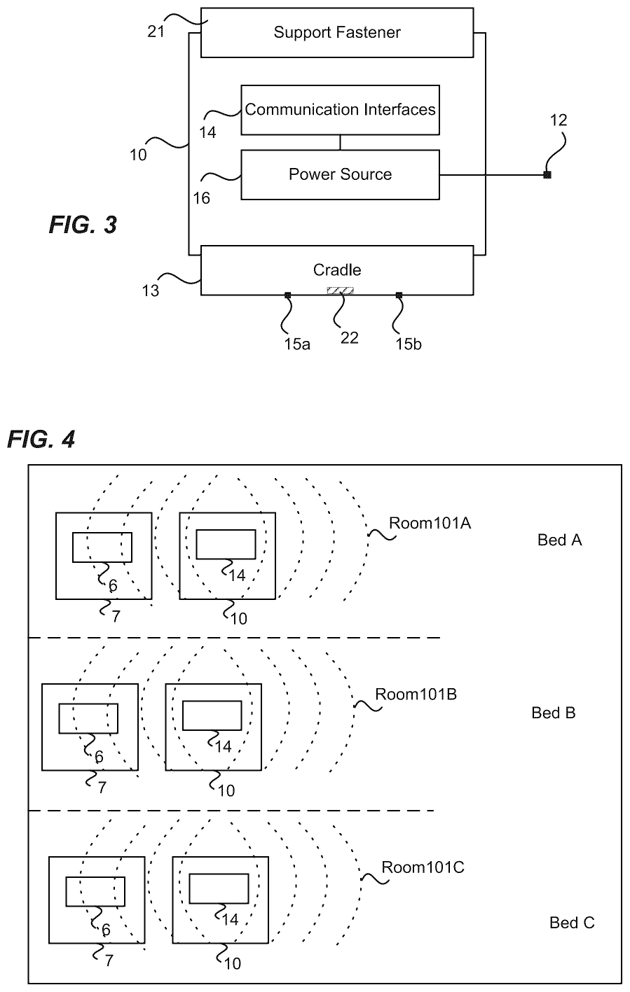

FIG. 3 illustrates a mount 10 (also referred to herein as a “simple mount”) for use in locations where wired network connectivity and/or power is not available, or when a lower-cost mount is desirable. The simple mount 10 may be used, for example, in emergency rooms, on transport beds, in low acuity units, and in ambulances. The simple mount 10 includes communication interfaces 14, a power source 16, a cradle 13, power contacts 15a, 15b, and a support fastener 21. Referring also to FIG. 2, the mount 10 may be secured to many different types of supports 20, such as a bed rail, an IV pole or a display mount. The mount 10 is preferably removably secured to a support 20 using the support fastener 21. The support fastener 21 can, for example, comprise a clamp, screw (or other fastener), magnetic contacts, an adhesive, or combinations thereof.

The communication interfaces 14 preferably includes at least two interfaces capable having different wireless communication protocols. The first interface preferably uses a low-power wireless link protocol, such as Bluetooth, Any, Zigbee, Wi-Fi Direct, or IrDa. The second interface preferably uses an electromagnetic induction communication protocol, such as RFID or NFC.

The first interface is configured to broadcast a signal indicative of a location (location signal) where the simple mount 10 is located. In order to reduce the power consumed by the communication interfaces 14, the location signal is preferably broadcast on a predetermined time interval. For many applications it may be desirable for time interval to be between 1 and 30 seconds and, more preferably, less than 15 seconds. Health care systems utilize the location of various devices, including patient monitors 7, to increase the operational functionality of the system. Accordingly, the communication interface 6 of the patient monitor 7 may be configured to receive the location signals that are periodically broadcast by the communication interfaces 14.

The first interface is preferably configured to broadcast the location signal only a short distance so that the location signal is only capable of being received by the patient monitor 7 that is either mounted to, or in very close proximity to, the simple mount 10. It is designed so that the location signal will not reach other patient monitors that are in the same vicinity. For example, in an emergency room multiple patients and their associated patient monitors 7 may be in close proximity to one another. If the location signal is broadcast too far it is possible that a different patient monitor or other devices could receive the location signal.

The first interface needs to broadcast the location signal at least as far as the distance between the communication interface 14 in the simple mount 10 and the communication interface 6 in the patient monitor 7 when the patient monitor 7 is mounted in the simple mount 10. As the exact distance the location signal is broadcast may vary based on different parameters, the first interface is preferably configured to broadcast the location signal a minimum distance that is greater than the distance between devices. However, the distance should be limited so that it could not be picked up by other devices. For example, the maximum distance the location signal should be broadcast should be less than a minimum distance a patient monitor associated with another patient could be. The maximum distance may be defined so that the patient monitor 7 would have to be on the patient 1 or the support 20 (i.e., bed) that the simple mount 10 was connected to. According to one embodiment, the distance that the location signal is broadcast may be less than 10 cm and, more preferably, less than 5 cm. This may mean that the location signal has a signal strength of no more than −98 dB at distance of 10 cm or, more preferably, at 5 cm.

In an exemplary implementation, the first interface may be a Bluetooth module that broadcasts the location signal at defined intervals (i.e., every 30 seconds). The time intervals for broadcasting the location signals may be programmable. Likewise, the location signal that is broadcast may be programmable. The location signal may be indicative of the location of the simple mount 10. The location signal may be defined in terms that are utilized by the health care system that the patient monitor 7 is communicating with. The location signal may utilize naming conventions that are consistent for all the devices communicating within the health care system. The location signal may identify, for example, room number (i.e., Room101B, 1174A), a department in a health care facility (e.g., ER, Admissions, Radiology, Unit2, Bed4, Floor2, Corridor7), an ambulance number (i.e., Ambulance14), or a predefined patient movement status (e.g., InTransit, WheelChair, Gurney16, TransportBed9).

The second interface may be used to pair the first communication interface with the patient monitor 7, so that communication is enabled between the first communication interface and the patient monitor 7.

The communication interface 14 may be programmed using a wireless device (i.e., laptop, cell phone) configured to program the communication interface 14. As noted, the programming may include assigning a location name for the location, the intervals at which the location signal is transmitted, and the broadcast range of the location signal. The communication interface 14 will need to be reprogrammed if the simple mount 10 is moved to a new location so that the location signal is accurate. For example, if the bed that the simple mount 10 is secured to is moved to another room the communication interface 14 will need to be reprogrammed to identify the new location. The location information for a particular simple mount 10 is then stored in the hospital network 11 (FIG. 1).

FIG. 4 illustrates a room in a health care environment having a plurality of beds according to at least one embodiment. The room may be room 101 and the beds located therein may be identified as Bed A, Bed B, and Bed C. Each of the beds has a simple mount 10 secured thereto and a patient monitor 7 docked therein. The communication interface (Bluetooth module) 14 associated with each bed is programmed with a location name (i.e., Room101A, Room101B, Room101C). The first interface of each communication interface 14 broadcasts the location name at defined intervals (i.e., every 30 seconds). The location name is broadcast a limited distance (i.e., no more than 10 cm) so that it can only be received by the patient monitor 7 that is docked thereto or located in close proximity thereto (i.e., clipped to patient, on bed). As is shown in FIG. 4, the distance the location names are broadcast cannot be received by patient monitors 7 located on other beds and associated with other patients. The communication interface (Bluetooth module) 6 within the patient monitor 7 receives the location signal broadcast from the simple mount 10 it is docked in. Once the patient monitor 7 obtains the location signal it communicates the location to the health care system. The health care system may use the location of the patient monitor 7 to take different actions.

Referring back to FIG. 3, the power source 16 may be an interface to be powered through an electrical outlet (not shown). The power source 16 provides power to the communication interface 14 and to patient monitor 7 when docked. The simple mount 10 and the patient monitor 7 may include power contacts 15a, 15b that are in communication with one another when the patient monitor 7 is docked in the simple mount 10. When the power contacts are in communication with one another, power is transferred from the power source 16 of the simple mount to the power source 9 of the patient monitor. The power contacts are such that inadvertent power transfers to other devices or individuals is prevented.

The simple mount 10 may also include one or more magnets 22 may be used by a patient monitor 7 to detect that it is docket in the simple mount 10. The magnets 22 may also be used to control the power contacts 15a, 15b—by only energizing them when the presence of a patient monitor 7 is detected. The patient monitor 7 may include a sensor, such as a Hall effect sensor, that produces a higher voltage when in close proximity to a magnet. Accordingly, if output voltage of the Hall effect sensor reaches a predetermined voltage, the patient monitor 7 could be programmed to indicate docking of the patient monitor 7 in a simple mount 10. Similarly if output voltage of the Hall effect drops below a predetermined voltage, the patient monitor 7 could be programmed to indicate undocking of the patient monitor 7 from a simple mount 10.

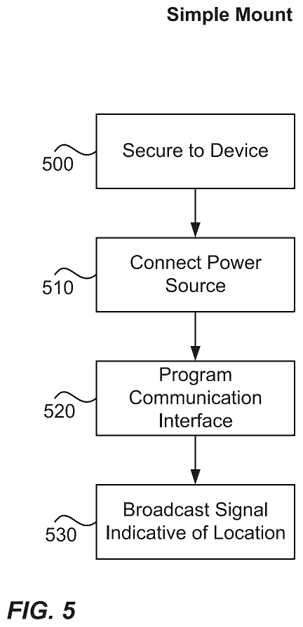

FIG. 5 is a flow diagram showing an illustrative example for programming and using the simple mount 10 to provide location information to the patient monitor 7. The simple mount 10 is secured to a support 20, such as a bed rail or IV pole (step 500), then connected to a power source 16 (step 510), such as a wall outlet. The location code for the simple mount 10 is then programmed into the communication interface 14 using a wireless device, such as a laptop computer (step 520). Programming of the communication interface 14 defines a location identifier to be broadcast by the communication interface 14 and, optionally, may determine the power at which the signal is broadcast and at what time interval the location signal will be broadcast. The communication interface 14 then broadcasts the location signal at the defined time interval (step 530). Steps 500 through 530 are preferably repeated any time the simple mount 10 is relocated to another support 20. Alternatively, the communication interface 14 could be programmed to broadcast a fixed, unique location identifier. In this case, the hospital network 11 may be adapted to associate that unique location identifier with a particular location.

When connected to a power source (step 510), the simple mount 10 may also be configured to selectively energize the power contacts 15a, 15b based on the detected presence of docked patient monitor 7. This could be accomplished, for example, using a magnetic switch or any other suitable means.

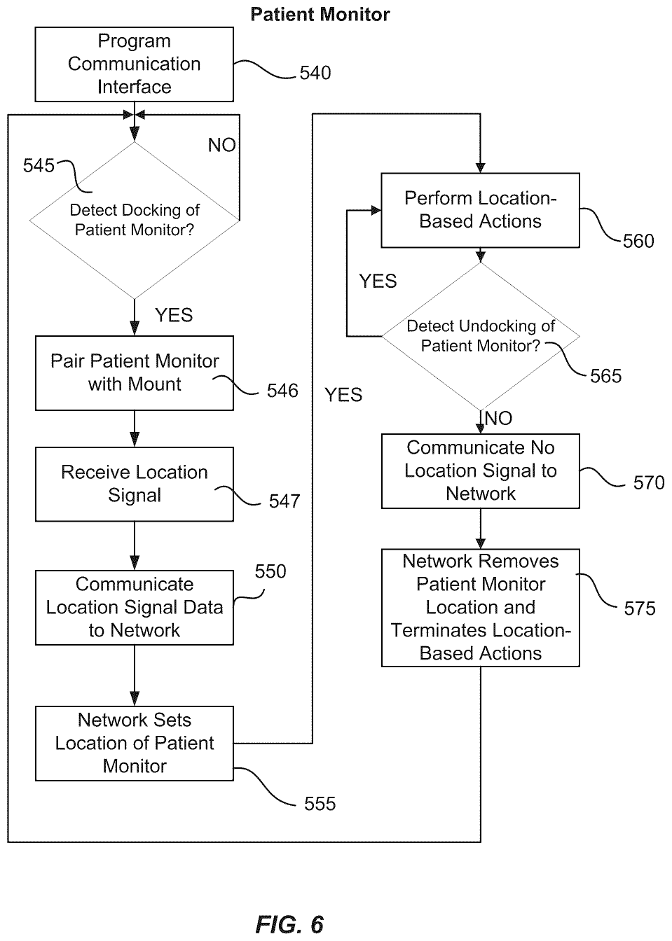

FIG. 6 is a flow diagram showing an illustrative example of using the location signal from the simple mount 10 to set the location of the patient monitor 7. The communication interface 6 of the patient monitor 7 is programmed to received and recognize the location signal (step 540). It should be noted that depending on the type of communication interface 6 that is utilized, the programming thereof to receive the broadcast location signals may limit the operation of the communication interface. For example, if the communication interface 6 includes a single Bluetooth module and the module is programmed to receive broadcast location signals it may not be available for other wireless communications via the Bluetooth module.

When the patient monitor 7 detects that it has docked with a simple mount 10 (step 545), it will attempt to pair with simple mount 10 (step 546) using the second interface (electromagnetic induction communication protocol). If the pairing is successful, the communication interface 6 will receive the location signal (step 547) through the first communication interface (Bluetooth) and communicate location data to the hospital network 11 (step 550). As noted above, this will typically occur when the patient monitor 7 is docked with the simple mount 10 that is transmitting the detected location signal. The hospital network 11 then identifies the location of the patient monitor 7 based on the detected location data (step 555). If any location-based actions are associated with that location, such location-based actions are then initiated (step 560). Examples of location-based actions include, adjusting parameters of the patent monitor, taking certain actions such as check in/out or billing, initiating a clinical action for a patient associated with the patient monitor, turning off an alarm, and setting a patient category.

As noted above, the patient monitor 7 preferably has the capability to detect that it has disengaged from the simple mount 10 (step 565). Until disengagement is detected, the hospital network 11 continues to identify the patient monitor 7 as being at that location and location-based actions continue. When disengagement is detected (step 565), the patient monitor 7 communicates to the hospital network 11 that no location signal is being received (step 570) and the hospital network 11 no longer identifies the patient monitor 7 as being at that location and terminates any location-based actions (step 575).

Optionally, the patient monitor 7 could be adapted to trigger an alarm if the patient monitor 7 is detected as being undocked and a sensed temperature of the patient monitor exceeds a predetermined temperature. Such an alarm is intended to alert clinical staff that the patient monitor 7 may be in contact with a patient and to avoid a potential burn or discomfort for the patient.

The patient monitor 7 may optionally be configured to require multiple consecutive detections of location signal (step 545) before communicating location data to the network (step 550), or multiple “no-detections” of a location signal (step 565) before communicating that it is no longer at a location (step 570). This could reduce the likelihood of an incorrect location identification based on the patient monitor being briefly placed in close proximity to a simple mount 10.

It should be noted that the steps illustrated are in no way intended to be limiting. The steps may be performed in a different order, steps may be added, steps may be deleted, steps may be modified, steps may be combined, and/or steps may be broken apart without departing from the current scope.

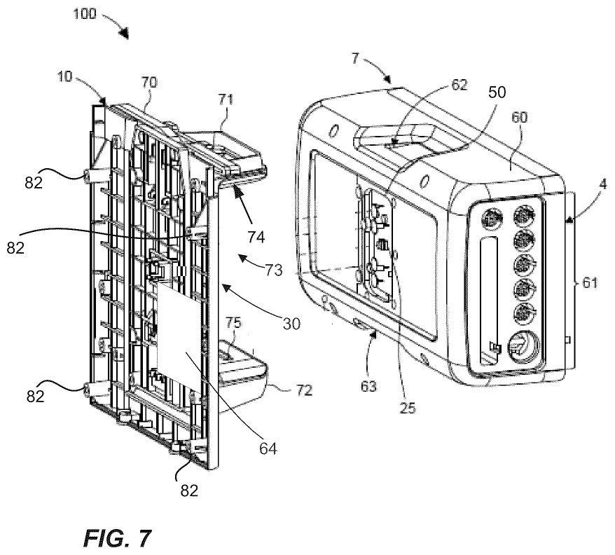

FIG. 7 is a perspective view of a docking system 100 according to one or more embodiments. The patient monitor 7 includes a housing 60 that includes a display 4 at its front face and a docking interface 50 at its rear face. The housing 60 also includes various connector ports 61 at a side face for being connected to various types of patient monitoring equipment and sensors 17 by wired connections 19. The housing 60 further includes interlocking recesses 62 and 63 located at opposing top and bottom sides of the housing 60 that are each configured to receive a docking arm of the simple mount 10 for docking.

The simple mount 10 includes a housing 70 that is configured receive the patient monitor 7 for docking. The housing 70 includes two docking arms 71 and 72 that extend outward and define a docking recess 73 configured to receive the patient monitor 7 therein. The housing 70 also includes the docking interface (not visible in FIG. 7) on a sidewall interposed between the two docking arms 71 and 72 that is outward facing and configured to engage with docking interface. The two docking arms 71 and 72 each include mechanical latches 74 and 75, respectively, that are configured to interlock with a corresponding interlocking recess 62 or 63 to secure the patient monitor 7 within the docking recess 73.

The docking interfaces 30, 50 may include power contacts that are engaged when the patient monitor 7 is docked in the simple mount to enable the simple mount 10 to provide power to the patent monitor 7. A Bluetooth module 64 is shown schematically in FIG. 7, which may be powered by a battery or external power supply available to the simple mount 10.

The backside of the simple mount 10 is simply illustrated as a plate having several screw holes 82 formed therein. The screw holes may be utilized to secure the simple mount to a support 20 with screws. The screw holes may also be utilized to secure clamps, brackets or the like that are then used to secure the mount to the device. According to other embodiments, the simple mount may be formed with different connectors on a back side thereof.

The ability of a simple mount 10 to provide location to the patent monitor at locations without network connectivity and, in some applications, without an external power source, allows the patient monitors 7 to be automatically associated with a location at locations that was not previously available. This may increase the ability of a health care system to use location to enhance the system. For example, the identification of an ambulance with patient data transmitted from the patient monitor in the ambulance to the hospital may ensure a smooth handoff from the EMTs to the ER staff and may expedite the admission of the patient. Likewise, knowing the patient is in transit based on the location information may provide for more efficient check-in/check-out of the patient.

While various embodiments have been disclosed, it will be apparent to those skilled in the art that various changes and modifications can be made which will achieve some of the advantages of the concepts disclosed herein without departing from the spirit and scope of the present disclosure. It will be obvious to those reasonably skilled in the art that other components performing the same functions may be suitably substituted. It is to be understood that other embodiments may be utilized and structural or logical changes may be made without departing from the scope of the present disclosure. It should be mentioned that features explained with reference to a specific figure may be combined with features of other figures, even in those not explicitly mentioned. Such modifications to the general inventive concept are intended to be covered by the appended claims and their legal equivalents.

Furthermore, the following claims are hereby incorporated into the detailed description, where each claim may stand on its own as a separate example embodiment. While each claim may stand on its own as a separate example embodiment, it is to be noted that—although a dependent claim may refer in the claims to a specific combination with one or more other claims—other example embodiments may also include a combination of the dependent claim with the subject matter of each other dependent or independent claim. Such combinations are proposed herein unless it is stated that a specific combination is not intended. Furthermore, it is intended to include also features of a claim to any other independent claim even if this claim is not directly made dependent to the independent claim.

It is further to be noted that methods disclosed in the specification or in the claims may be implemented by a device having means for performing each of the respective acts of these methods. For example, the techniques described in this disclosure may be implemented, at least in part, in hardware, software, firmware, or any combination thereof. For example, various aspects of the described techniques may be implemented within one or more processors, including one or more microprocessors, DSPs, ASICs, or any other equivalent integrated or discrete logic circuitry, as well as any combinations of such components.

Further, it is to be understood that the disclosure of multiple acts or functions disclosed in the specification or in the claims may not be construed as to be within the specific order. Therefore, the disclosure of multiple acts or functions will not limit these to a particular order unless such acts or functions are not interchangeable for technical reasons. Furthermore, in some embodiments a single act may include or may be broken into multiple sub acts. Such sub acts may be included and part of the disclosure of this single act unless explicitly excluded.

1. A method of associating a location with a patient monitor having a monitor housing, a graphical user interface, a power source, a communication interface capable of communicating using a wireless communication protocol, and a physiological sensor interface, the method comprising:
(a) providing a first mount having a first mount housing, first cradle, a first mount power source, and a first wireless communication interface, the first cradle being adapted to removably secure the patient monitor;
(b) broadcasting a first location signal using the first wireless communication interface, the first location signal having a signal strength of no more than −98 dBm at a distance of 10 cm from the first mount housing, the first location signal including location data that is associated with a first location and the first mount by a hospital network; and
(c) associating the patient monitor with the first location when the patient monitor receives the first location signal.

2. The method of claim 1, further comprising:
(d) pairing the first mount with the patient monitor using a second wireless communication interface.

3. The method of claim 2, wherein the second communication interface uses an electromagnetic induction protocol.

4. (canceled)

5. The method of claim 1, wherein step (c) further comprises communicating the received first location signal to the hospital network, wherein the patient monitor is associated with the first location by the hospital network.

6. The method of claim 1, further comprising:
(e) after performing step (c), initiating at least one location-based action based on the first location.

7. The method of claim 6, wherein the at least one at least one location-based action is selected from the group of: adjusting an operating parameter of the patient monitor, initiating a clinical action for a patient associated with the patient monitor, turning off an alarm, and setting a patient category.

8. (canceled)

9. The method of claim 1, further comprising:
(f) after performing step (c), disassociating the patient monitor with the first location when the first location signal is not received by the patient monitor for a first predetermined period of time.

10. The method of claim 1, further comprising, performing step (b) at a predetermined time interval.

11. The method of claim 1, wherein the first location is selected from the group of: a room number, a department of a health care facility, an ambulance number, or a predefined patient movement status.

12. The method of claim 1, wherein step (c) comprises associating the patient monitor with the first location when the patient monitor receives the first location signal a predetermined number of times, the predetermined number of times being greater than one.

13. The method of claim 1, further comprising:
(h) providing electrical power from the first mount to the patient monitor through a plurality of first mount power contacts located within the first cradle that are adapted to engage monitor power contacts located on the monitor housing when the patient monitor is mounted in the first cradle.

14. (canceled)

15. The method of claim 1, further comprising:
(i) providing a second mount having a second mount housing, second cradle, a second power source, and a second wireless communication interface, the second cradle being adapted to removably secure the patient monitor;
(j) broadcasting a second location signal using the second wireless communication interface, the second location signal having a signal strength of no more than −98 dBm at a distance of 10 cm from the second mount housing, the second location signal including location data that is associated with a second location by the hospital network; and
(k) associating the patient monitor with the second location when the patient monitor receives the second location signal.

16.-26. (canceled)

27. A method of associating a location with a patient monitor having a monitor housing, a graphical user interface, a power source, a first monitor communication interface, a second monitor communication interface, and a physiological sensor interface, the method comprising:
a. providing a first mount having a first mount housing, first cradle, a first mount power source, a first mount wireless communication interface, and a second mount wireless communication interface, the first cradle being adapted to removably secure the patient monitor, the first mount wireless communication interface and the first monitor communication interface using a first wireless communication protocol, the second mount wireless communication interface and the second monitor communication interface using a second wireless communication protocol, the second wireless communication protocol being an electromagnetic induction communication protocol;
b. broadcasting a first location signal at a first predetermined interval using the first wireless communication interface, the first location signal including location data that is associated with a first location and the first mount by a hospital network;
c. detecting that the patient monitor is docked to the first cradle;
d. when the patient monitor is detected as being docked in the first cradle, pairing the first mount communication interface with the first monitor communication interface using the second wireless communication protocol; and
e. associating the patient monitor with the first location if the location data is received by the first monitor communication interface from the first mount communication interface a first predetermined number of times.

28. The method of claim 27, further comprising:
(f) disassociating the patient monitor from the first location if, after performing step (e), the first location signal is not detected by the first monitor communication interface a second predetermined number of times.

29. The method of claim 27, further comprising:
(g) disassociating the patient monitor from the first location if, after performing step (e), the patient monitor is detected as being undocked from the first cradle.

30. The method of claim 27, wherein the first communication protocol is a Bluetooth communication protocol.

31. The method of claim 27, wherein the second communication protocol is selected from the group of RFID and NFC.

32. The method of claim 27, wherein the patient monitor is detected as being docked to the first cradle when an output voltage of a first hall effect sensor reaches a first predetermined docking voltage.

33. The method of claim 32, wherein the patient monitor is detected as being undocked from the first cradle when the output voltage of the first hall effect sensor drops below a second predetermined docking voltage.

34. The method of claim 27, wherein the first location signal has a signal strength of no more than −98 dBm at a distance of 10 cm from the first mount housing.
